(19) United States
(12) Patent Application Publication
(10) Pub. No.: US 2012/0116155 A1
(43) Pub. Date: May 10, 2012

(54) LIGHT-BASED, TRANSCUTANEOUS VIDEO SIGNAL TRANSMISSION

(75) Inventor: Robert M. Trusty, Cincinnati, OH (US)

(73) Assignee: Ethicon Endo-Surgery, Inc., Cincinnati, OH (US)

(21) Appl. No.: 12/939,441

(22) Filed: Nov. 4, 2010

(51) Int. Cl. A61B 1/06; A61B 1/04

(52) U.S. Cl. 600/109; 600/178

(57) ABSTRACT

A surgical device is disclosed which includes an optical source for wirelessly transmitting a light based signal transcutaneously and a receiver for receiving the light based signals. The wireless coupling of signals between the optical source and the receiver wirelessly transmits video images from an internal site in a patient to a video monitor or other viewer outside the patient, and may wirelessly transmit control signals from a controller outside of the patient to an instrument inside the patient during a therapeutic or diagnostic surgical procedure.

### BACKGROUND

i. Field of the Invention

The present application relates to methods and devices for use in medical procedures, including without limitation, minimally invasive surgical and diagnostic procedures and, more particularly, to devices for wirelessly transmitting video images through living tissue using an optical signal carrier.

ii. Description of the Related Art

In minimally invasive medical procedures, such as laparoscopic surgery, a surgeon may place one or more small ports into a patient's abdomen to gain access to the abdominal cavity of the patient. Surgical and diagnostic instruments are delivered into the patient's body via one or more ports. A surgeon may use, for example, a port for insufflating the abdominal cavity to create space, a port for introducing a laparoscope for viewing, and a number of other ports for introducing surgical instruments for operating on tissue. Other minimally invasive surgical procedures include natural orifice transluminal endoscopic surgery (NOTES™) wherein surgical instruments and viewing devices are introduced into a patient's body through, for example, the mouth, vagina, nose, or rectum. Another class of such minimally invasive surgery includes magnetically-based, (MAGS) devices. MAGS devices typically include an internal device that provides therapy to the patient (e.g. electro-cautery) or information to the surgeon (e.g. video camera) and an external magnet used by the surgeon to control the internal device.

Some of the instruments delivered through a port may be electronic in nature and require electronic data signals to be delivered to them to operate, for example to adjust the focus of a lens system. They may also need to deliver electronic information signals in the other direction to personnel in the operating room, for example an encoded video stream from a camera for display in the Operating Room. Currently, signals generated by and sent to conventional instruments are transmitted in and out of the patient via a hardwired electronic tether. Alternately, signals are also transmitted in and out of the patient via a wireless Radio Frequency (RF) link.

The foregoing discussion is intended only to illustrate various aspects of the related art in the field of the invention at the time, and should not be taken as a disavowal of claim scope.

### SUMMARY

While using tethers or an RF link to carry signals across a patient's tissue is an acceptable technique, it would be preferable to minimize or eliminate the tether and increase the bandwidth and eliminate an RF signal altogether. Disclosed herein is a means to wirelessly couple signals in at least one direction between an optical source and a receiver through a patient's tissue during a therapeutic or diagnostic surgical procedure.

More particularly, there is described an apparatus that includes an optical source for wirelessly transmitting a light based signal transcutaneously and a receiver for receiving the light based signal. The optical source may comprise at least one, and preferably a plurality of light emitting diodes or laser sources.

In one embodiment, the optical source may emit light at a wavelength between 400 nm and 15,000 nm, above the ultraviolet range and below the far infrared range of the electromagnetic spectrum (CIE scale). In other embodiments, the optical source may emit light at a wavelength between 400 to 3000 nm, and preferably at wavelengths between 700 to 1400 nm (near infrared). In still another embodiment, the optical source may emit light at wavelengths between 750 to 1100 nm. The receiver may include at least one filter for controlling the wavelength of the received light based signals.

The apparatus may further include an external unit for positioning, in use, on an external surface of a patient, and an internal unit for positioning, in use, adjacent tissue in an internal body cavity of the patient.

The external unit may have at least one said optical source and the internal unit may have at least one said receiver for receiving the light based signals from the external unit's optical source.

Alternatively, the internal unit may have at least one optical source and the external unit may have at least one receiver for receiving the light based signals from the internal unit's optical source. In yet another embodiment, the external unit may have both the optical source and a receiver, and the internal unit may have both the receiver for and an optical source.

The external unit may have a plurality of receivers and may further include a plurality of optical sources for wirelessly transmitting light based signals transcutaneously. The internal unit may have a plurality of optical sources and may further include a plurality of receivers. Each of the plurality of receivers may be optically configured for receiving the light based signals from a different one of the plurality of optical sources to define optically coupled pairs. There may be, for example, four optically coupled pairs.

In certain embodiments, such as those intended for MACS applications, each of the internal and external units include a magnet positioned for magnetically attracting the magnet of the other of the external and internal units, such that manipulation of the external unit controls the positioning of the internal unit within the body cavity. In another embodiment, each of the internal and external units has two magnets of opposing magnetic polarity.

In a preferred embodiment, the apparatus may include a working instrument operatively connected to the internal unit, such as an imaging device, or video camera, for generating video signals, wherein the light based signals emitted from the plurality of optical sources of the internal unit are video signals encoding a video image. The plurality of receivers on the external unit may be operatively connected to a video viewer for displaying the video image. The light based signals emitted from the external unit may be control signals for controlling the working instrument, such as controls for controlling a video camera.

A method is described for wirelessly transmitting a light based signal transcutaneously. The method includes inserting an internal unit into an internal body cavity of a patient undergoing a medical procedure, positioning the internal unit adjacent tissue in an internal body cavity of the patient, and positioning an external unit on an external tissue surface of the patient opposite the position of the internal unit. The external unit used in the method may have at least one of (i) an optical source for wirelessly transmitting a light based signal transcutaneously and (ii) a receiver for receiving a light based signal transcutaneously, and the internal unit used in the procedure may have at least one of (i) a receiver for receiving light based signals from the external unit optical source, and (ii) an optical source for transmitting the light based signals to the external unit receiver. The method further includes transmitting light based signals from the external unit optical source to the internal unit receiver to effect operation of a working instrument operatively connected to the internal unit, and transmitting light based signals from the internal unit optical source to the external unit receiver to communicate information, such as video image signals, from the internal body cavity of the patient to a controller on the exterior of the patient.

Associated software and electronics in each of the internal and external units may be provided to optimize the imaging and controls.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 shows a prior art standard visualization module with a transcutaneous electronic tether deployed in a patient.

FIG. 2 shows an external unit having at least one magnet with multiple optical sources and multiple receivers for transmitting and receiving light-based electromagnetic signals, respectively.

FIG. 3 shows an internal unit having at least one magnet with multiple optical sources and multiple receivers for transmitting and receiving light-based electromagnetic signals, respectively.

FIG. 4 shows a composite of the system having the external magnetic unit of FIG. 2 and an internal magnetic unit and working instrument in operation transmitting signals back and forth across the abdominal wall.

FIG. 5 shows a perspective view of the system of FIG. 4 with the external unit receiving the light-based electromagnetic signals from the internal unit.

FIG. 6 shows a view of the receivers of the external unit receiving signal transmission from the internal unit.

FIG. 7 shows a view of the internal unit with a working instrument mounted therein transmitting signals to the external unit of FIG. 6.

FIG. 8 shows a view of the external unit transmitting signals to the internal unit to control the working instrument mounted in the internal unit.

FIG. 9 shows a view of the system having with the external magnetic unit connected to a controller and a monitor and the internal magnetic unit having an alternative working instrument actuated by signals transmitted across the abdominal wall.

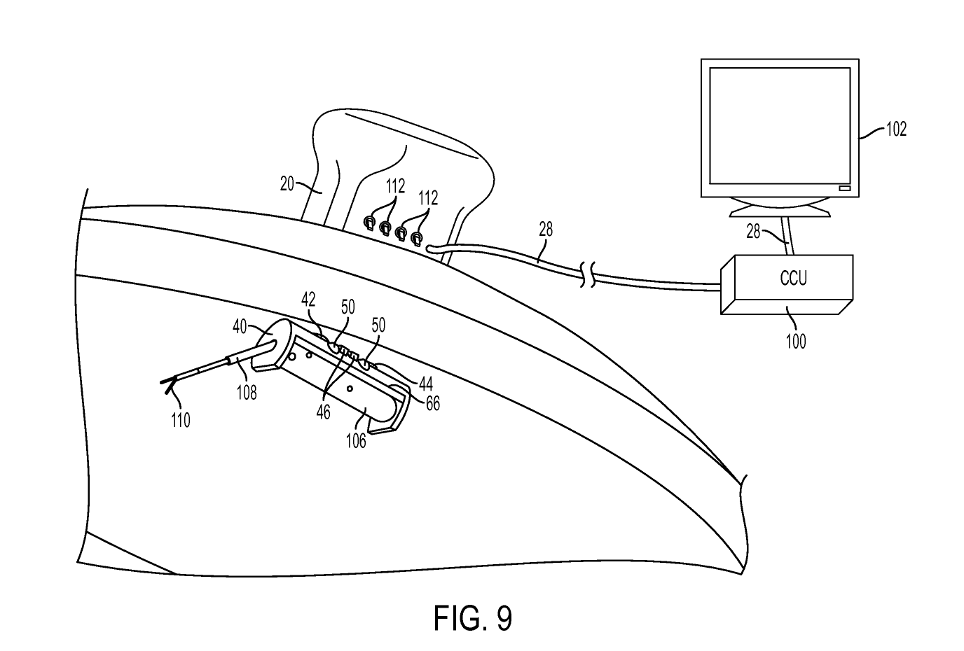

FIG. 10 is a graph of the absorption coefficient v. wavelength for various biological components.

FIG. 11 is a graph of transmission percentage v. wavelength for hemoglobin, fat and water.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments.

Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located farthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

As used herein, the term “patient,” refers to any human or animal on which a medical procedure, such as a surgical, therapeutic, or diagnostic procedure, may be performed. As used herein, the term “internal site” of a patient means a lumen, body cavity or other location in a patient's body including, without limitation, sites accessible through natural orifices or through incisions.

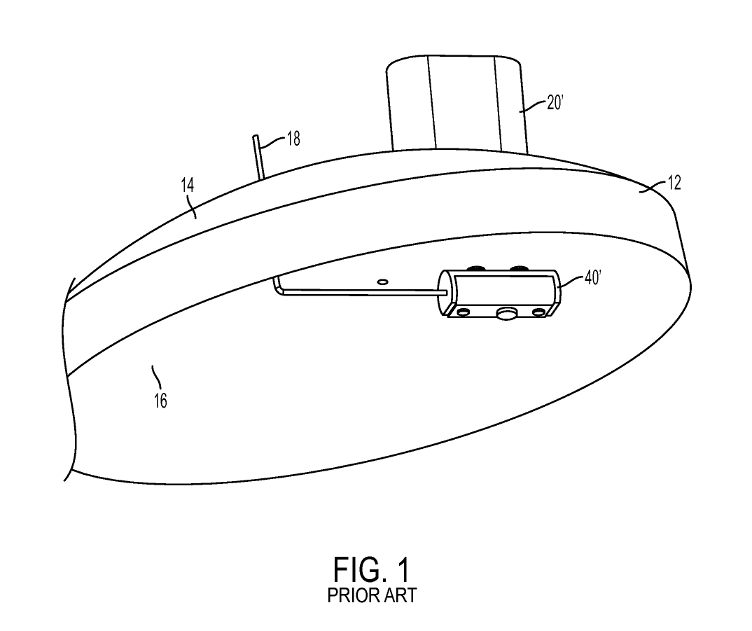

Heretofore, video signals from an internal camera have been transmitted through living tissue 12 of a patient undergoing a therapeutic or diagnostic surgical procedure through a tether 18 that passes from the camera 40′ through the tissue 12 to a video receiver and viewer or monitor (not shown) on the outside of the patient. In a MACS system, as shown in FIG. 1, an external control unit 20′ having one or more permanent magnets or electro-magnetic magnets housed therein is positioned on the outer surface 14 of the patient's body. The camera is carried in an internal magnetic sled 40′ having its own magnets, which are attached to the external control unit 20′. Movement of the external control unit 20′ moves the internal magnetic sled 40′ and the camera carried in the sled. Video images captured by the camera are carried by the tether 18 outside of the body for viewing by the clinician or surgeon. Alternately, the camera and magnetic sled may be integrated into a single unit.

The wireless coupling of signals described herein may be used to wirelessly transmit video images during a therapeutic or diagnostic surgical procedure. In one embodiment, a camera obtains a video image of a structure or location inside the patient's body and transmits the encoded video signal wirelessly by optical sources, such as via laser or light emitting diodes (LEDs), across the body wall to one or a plurality of receivers on the external unit. The optical sources emit electromagnetic radiation in the form of a beam in the desired wavelength range and modulate the beam by switching it on and off rapidly (digital) or continuously modulating the signal amplitude (analog), to encode data. Communication may be serial wherein one light blinks on and off quickly to generate a 1,0 form of signal which carry the video signals at megahertz speeds with the plurality of optical sources. The communication of video signals across the body wall to the receivers can be faster by using the optical sources in parallel, with the plurality of optical sources arranged in multiple lines of transmission occurring simultaneously.

Transmission of signals may be in the opposite direction, from outside of the patient to the internal unit inside the patient. Signals may be transmitted from a control panel by electromagnetic communication to transmitters on the external control unit which transmits signals to one or a plurality of receivers on the internal unit, which are communicated to a working instrument, such as a camera. The signals may include, without limitation, signals to adjust the focus of the camera lens, to control a zoom lens, to change the direction of the viewer or to change the direction or intensity of light. Signals to control other kinds of working instruments may also be transmitted, such as a signal to open or close the arms of a grasper or pivot an end-effecter or activate an energy-based surgical device. Electromagnetic and specifically, radio frequency (RF) wireless transmission of video signals is well understood.

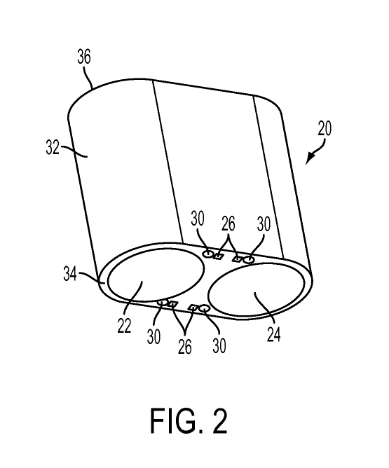

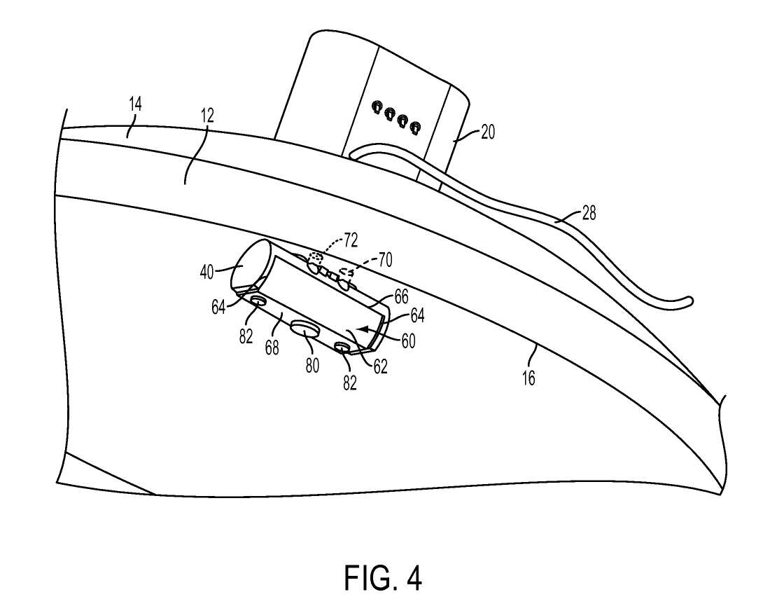

The light based signaling system described herein includes an external unit 20 and an internal unit 40. One of the units 20 or 40 has at least one optical source for sending light based signals through living tissue and the other unit, 20 or 40, has at least one receiver for receiving the light based signals. Referring to FIG. 2, the embodiment of the external unit 40 shown includes a housing 32, north and south permanent magnets 22, 24 housed in the housing 32, at least one and preferably a plurality of optical sources 30 and at least one and preferably a plurality of receivers 26. The distal surface 34 of the external unit 20 that in use would be in contact with the patient's external body surface 14 (see FIG. 4) includes the optical sources 30, receivers 26 and the facing surfaces of magnets 22, 24. The proximal surface 36 of the external unit 20 is structured to be held by a surgeon or clinician for movement across the surface 14 of the patient.

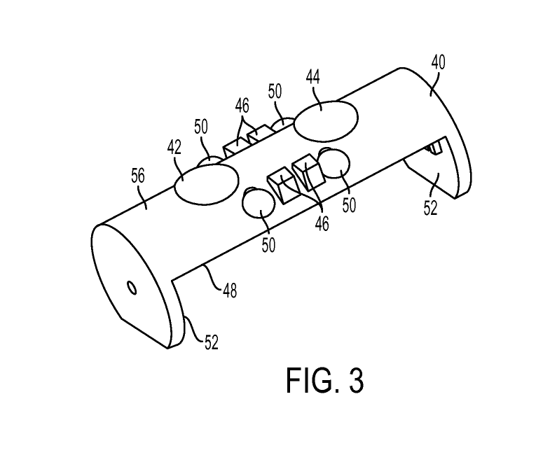

An embodiment of an internal unit 40 is shown in FIG. 3. Internal unit 40 may be structured to include an external proximal surface 56 that in use would face, and be positioned adjacent to and in contact with, the internal body surface 16 of the patient. Unit 40 also is shown having end walls 52 and an internal cavity 48 which together define a space in which a surgical or diagnostic camera or similar working instrument may be mounted. Suitable known attachment means are provided to secure the working instrument into the cavity 48.

The embodiment of the internal unit 40 shown in FIG. 3 includes on surface 56 north and south magnets 42 and 44, optical sources 50, and receivers 46.

The external unit 20 may have a plurality of receivers 26 and a plurality of optical sources 30 for wirelessly transmitting light based signals transcutaneously. Similarly, the internal unit 40 has a plurality of optical sources 50 and a plurality of receivers 46. Each of the plurality of receivers 26/46 is optically configured for receiving the light based signals from a different one of the plurality of optical sources 30/50 to define optically coupled pairs. There are four optically coupled pairs shown in the figures, but those skilled in the art will recognize that more or less may suffice.

The receivers 26 transmit the video signals over a telecommunications link or relays the signals over a hard wired link to a camera control unit (CCU) 100 which uses the received video signals to generate the video images for display on a monitor 102 for viewing by the clinician. Any suitable known receivers, optical sources, computer relays, monitors and software for processing the signals may be used.

For example, the components necessary for a single optical link to transmit a video signal and show it on an operating room display include a camera/illumination unit, a signal processing circuit, an LED or Laser that is connected to and modulated by the signal processing circuit with a lens on the output that aims and focuses the optical beam to an appropriate diameter for collection by the receiving unit, a housing, a receiver/transmitter unit, a CCU 100, appropriate cabling (e.g., 28), and a display unit 102.

An embodiment of a camera/illumination unit may include an electronics board with standard off-the-shelf components such as power supplies, resistors, capacitors, integrated circuits, logic components, signal processing components, software and the like, a light source, such as a white light LED with a concentrating/focusing lens to direct the light onto the target tissue of interest, and a camera head that includes an appropriate lens system for collecting and focusing an image of the tissue of interest onto a multi-pixel Charge Coupled Device (CCD) or Complimentary Metal Oxide Semiconductor (CMOS) array connected to the electronics board.

Alternately, the camera/illuminator unit may be configured to collect two images appropriate for later display in 3D. In this case, two parallel units would be consolidated within one housing and two signals would be broadcast via the light link.

The signal processing circuit may include an electronics board similar to the board used in the camera/illumination unit that accepts the video signal output of the camera/illumination board and converts it to an appropriate drive signal for an LED or Laser.

The housing encases the electronics and may include one or more optically transparent windows. The housing should be shaped so as not to cause damage to any tissue it comes into contact with. The optically transparent windows may also act as an optical filter to narrow the optical band of the transmitted signal. Alternately, the filter may be included directly on the output lens of the optical source.

In yet another embodiment, the optical filtering may be accomplished by use of a receiver/transmitter unit.

The receiver/transmitter unit may include a housing with an optical window that is shaped so as not to cause damage to any tissue it comes into contact with, a detector with a collection lens and, potentially an optical filter, one or more circuit boards similar to those described above which is designed to accept the signal output from the detectors and filter and encode it appropriately and transmit it either directly to a standard Operating Room display or to a CCU.

The CCU 100 may accept as input the output of the receiver/transmitter unit described above, and may include power supplies and circuit boards designed to de-encode the video signal and convert it to a signal that is appropriate for a standard Operating Room display.

Cabling is typically used to connect the receiver/transmitter unit directly or indirectly to the display or to the CCU. Alternately, the receiver/transmitter unit may wirelessly broadcast the signal to a CCU. In this case, the receiver/transmitter unit would include an additional circuit board, similar to those described above, designed to convert the output to a wireless (typically RF) signal at an electronic frequency appropriate to medical applications and broadcast the signal to a receiver in the CCU.

The display unit 102 may be a standard operating room display, such as a flat-screen LCD, plasma display, or cathode ray tube display or an equivalent means of viewing the video images.

The components necessary for a single optical link to transmit a signal originating from outside the patient that results in an action on the internal unit are very similar to those described above, except reversed. The end-action would be, for example, turning a stepper motor on to move a lens in front of the CCD or CMOS array to change the zoom or focus or, alternately, would be a stepper motor used to open the jaws of a grasper, or alternately, could be closing a switch that energizes a harmonic scalpel end effector or monopolar electrocautery unit. The signal input might be a button mounted on the external control unit 20 magnet, whose action would be transmitted across the light link.

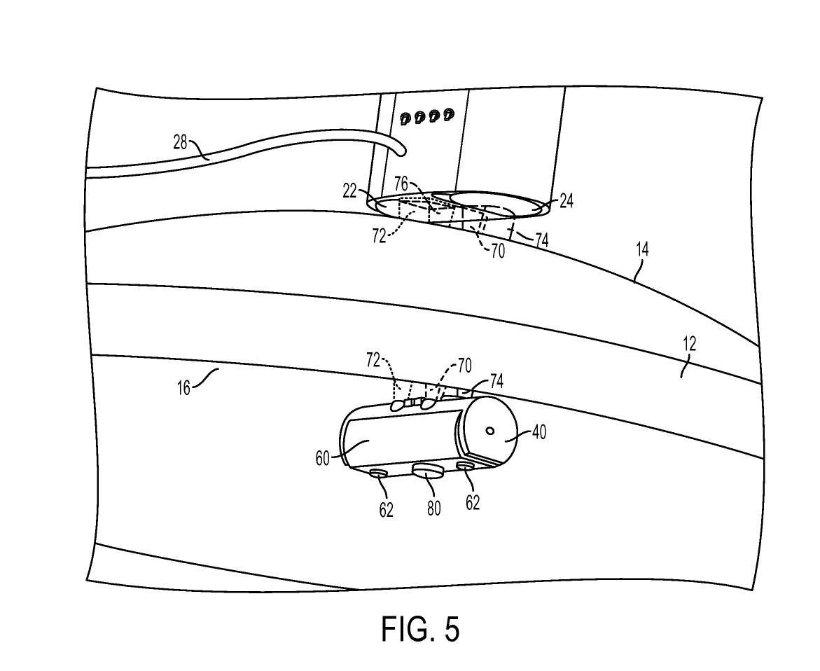

FIGS. 4 and 5 are illustrative of the external unit 20 and internal unit 40 in use. The patient's body, for example the abdominal or pelvic wall, is represented by wall 12 having outer surface 14 and an interior surface 16 of a body cavity. External unit 20 may be battery powered or may be connected by at least one tether 28 to a power source. Tether 28 or a second tether may connect external unit 20 to a video monitor 102, a control unit or a computer 100. Internal unit 40 is shown with a working instrument, such as camera 60, within the cavity 48. Camera 60 includes body 62, ends 64 held between end walls 52 of internal unit 40, proximal side 66 and a distal facing side 68. Side 68 includes a lens or window 80 for protecting the camera or capturing images of the tissue of the patient and light sources 82 for lighting the field of tissue for viewing and video capture.

The camera lens 80 views images of tissue. The video signals are transmitted by light energy beamed from LEDs 50 in the form of light cones 70, 72, 74, and 76 through the body wall 12 to receivers 26 on the distal surface of external unit 20. The video signal received by receivers 26 are communicated by a tether 28 or by wireless signals, such as radio frequency signals, to a video screen 102, CCU or a computer 100. Magnets 22, 24 of external unit 20 align with magnets 44, 42 of internal unit 40 to keep LEDs 30 aligned with receivers 46 and LEDs 50 aligned with receivers 25.

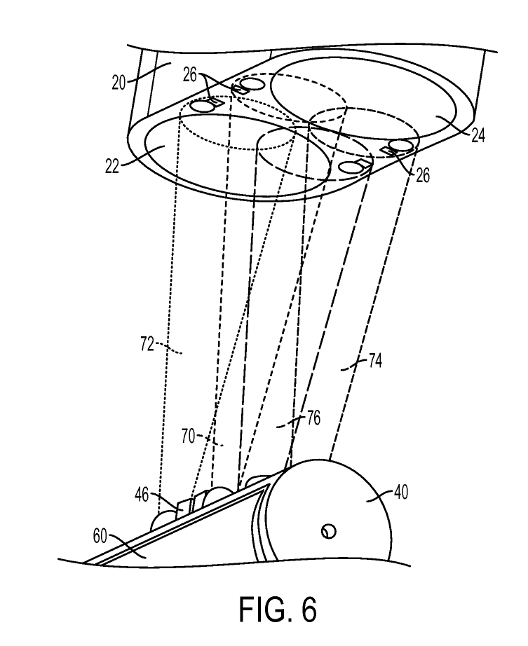

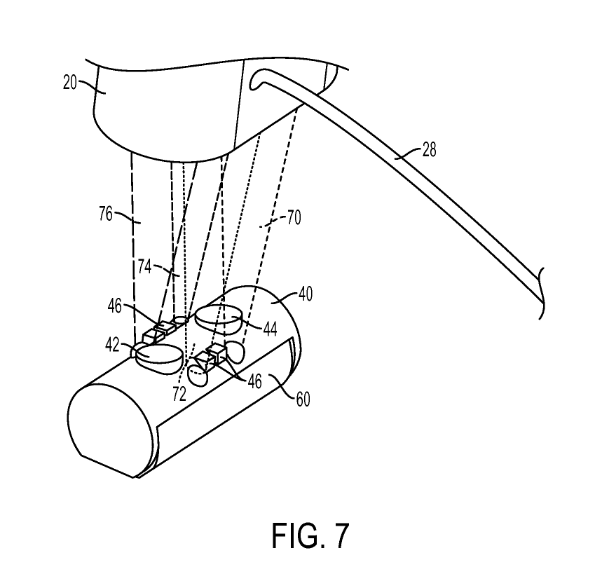

FIGS. 6 and 7 show the light based transmission of video signal beams 70, 72, 74, 76 from each of four LEDs 50 or internal unit 20 to each of four receivers 26 on external unit 40.

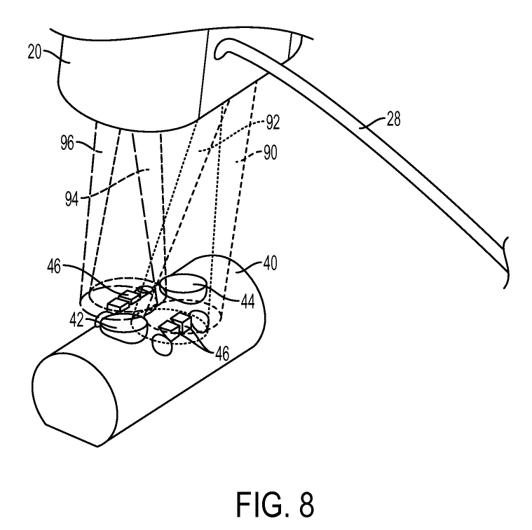

FIG. 8 shows the light based transmission of command signal beams 90, 92, 94, 96 from each of four LEDs 30 on external until 20 to each of four receivers 46 on internal unit 40.

Light sources such as LEDs and lasers can operate at high signal bandwidth and at the wavelength appropriate for the intended application (e.g., near infrared) so as to be able to transmit analog video signals through patient's tissue (e.g. from the peritoneal cavity across the abdominal or pelvic wall) to a receiver on or near the exterior surface of the patient according to established standards, such as the National Television Standards Committee (NTSC), the phase alternating lines (PAL), or sequential color with memory (SECAM). NTSC or PAL video signals currently utilize two electrical conductors, one positive (+) signal and the other ground or negative (−) signal. The LEDs or lasers would replace the positive (+) signal leg, and due to the nature of optical transmission, no negative (−) signal is needed. Having two-way capability allows the external unit to send control commands back to the internal unit 40 to the patient that could, for example, cause the device to focus, or zoom, or turn a motor on, or fire a staple, or open a grasper.

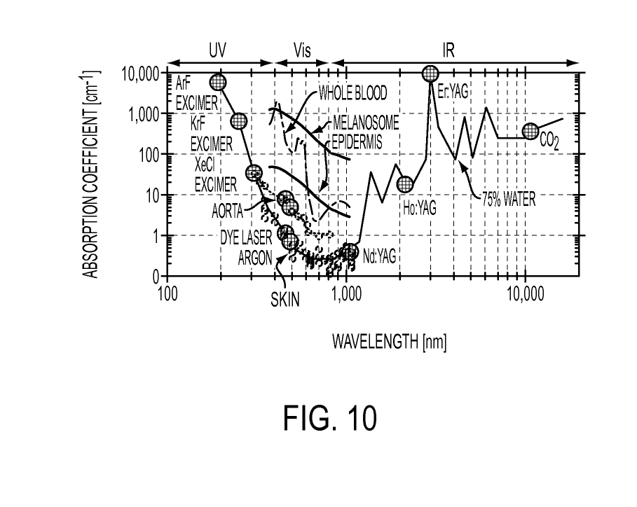

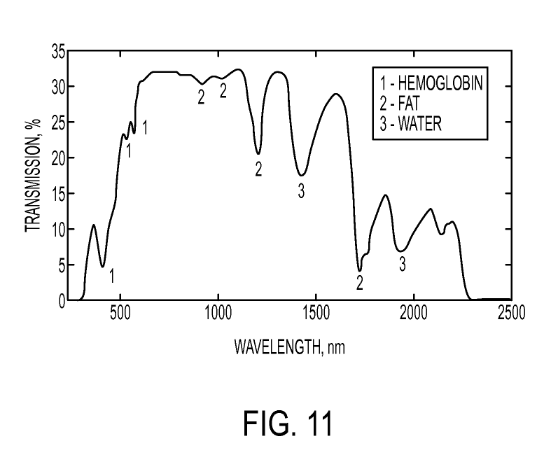

In one embodiment, the optical source may emit light at a wavelength between 400 nm and 15,000 nm, above the ultraviolet range and below the far infrared range of the electromagnetic spectrum (CIE scale). In other embodiments, the optical source may emit light at a wavelength between 400 to 3000 nm, and preferably at wavelengths between 700 to 1400 nm (near infrared). The wavelength of light signal beams are preferably in the range of 750 to 1100 nm, but any other wavelength of light will suffice provided the receivers 26, 46 are coordinated to receive the signals and the signal power is strong enough to pass through the patient tissue without causing harm. Those skilled in the art will recognize that long and short wavelengths would be absorbed by the tissue, so that far infrared (greater than 15,000 nm) and ultraviolet (less than 400 nm) wavelengths will not work. It is also known that there are water absorption bands, for example around 1310 nm and elsewhere, and that these portions of the spectrum would preferably be avoided. As shown in FIGS. 10 and 11, the optical absorption coefficient is minimized and transmission is maximized in the wavelength region from 600 nm-1000 nm for many human tissue and fluid types. As there are also many optical sources available in these bands, it would be preferable, but not required, to operate within this band

In an alternative embodiment, the internal unit 40 and camera 60 may be a single integral device rather than the distinct modules shown in FIGS. 4-7. For example, the embodiment shown in FIG. 3 or an embodiment without side walls 52 may include a lens system and light source.

Alternatively, a conventional MACS surgical camera may be equipped with signal transmission LEDs 50 and receivers 46. The conventional electronic components may be provided on a circuit board with battery and processor chips.

The external unit 20 may be equipped only to receive light based signal transmissions (70, 72, 74, 76) to receivers 26 from LEDs 50 but preferably is equipped both to receive light based signals and to send light based signals (90, 92, 94, 96) from LEDs 30 to internal unit 40 receivers 46. The signals transmitted from LEDs 30 to receivers 46 may be command signals containing instructions for maneuvering internal unit 40 camera 60, or another working instrument mounted in cavity 48, such as a mechanical end-effector 106 shown in FIG. 4. The end-effector may be a grasper having an arm 108 that may be actuated to swing outwardly away from the unit 40 or swing in towards unit 40 or to adjacent tissue. The end of the grasper has jaws 110 that may be opened and closed by triggering a switch control 112 on external unit 20 that initiates transmission of command signals to receivers 46 of internal unit 40, and forwarded to end-effector 106 circuits to control the arm 108 and jaws 110.

External unit 20 may also include control buttons with, for example, rocker switches, to activate a command signal transmission to internal unit 40.

All of the LEDs 30 or 50 may emit light of the same wavelength or each may emit light of a different wavelength or range of wavelengths from the other LEDs 30 or 50 on the same unit 40 or 20, or narrow band-pass filters may be employed on source/receiver pairs to isolate the signals from on another. Alternately, the source/receiver pairs may be located physically such that no other detector can “see” unintended sources. Alternatively, the transmissions may be adjusted or timed so that only one or a pair of LEDs send transmissions at a time or within desired intervals. Alternatively the receivers 26, 46 can be adjusted so that only one or a pair of receivers can receive light transmissions at once or during an interval. Optionally, the receivers may include filters to separate or exclude wavelengths of a particular range. Alternately, the signals may exist on a carrier frequency, similar to an FM broadcast signal format in the RF band, and each source/detector pair may operate on its own carrier frequency, thus isolating the signals from one another.

In an embodiment having a relatively small internal unit 40 for use, for example, in limited spaces within a patient's body, a plurality of LEDs may be used where each LED transmits light at a different wavelength or is optically filtered appropriately. The receivers 26 on external unit 20 would reflect away all light not within the desired range for such receiver 26. The light emissions can thus be optically isolated from each other so the overlapping cones of emitted light do not overlap when received. The isolated light signals can be used for parallel communications.

A small array of LEDs, such as the 4×4 arrays shown in the figures, may be arranged on a chip in each unit 20/40. A corresponding 4×4 array of receivers may be arranged on the opposing unit 40/20. The array of receivers 26/46 should not each detect light from each of the LEDs. The receivers 26/46 are structured or equipped with filters so that a receiver receives light only from its paired LED on the opposite unit. The filters on each LED absorb or reflect all light except the light within the wavelength range meant for that receiver.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions.

Endoscopic minimally invasive therapeutic or diagnostic surgical medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small—keyhole—incision incisions (usually 0.5-2.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam. Alternately, the device may be of a single-use disposable nature, and would be delivered sterilized and disposed of after a procedure.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An apparatus comprising:
an external unit for positioning, in use, on an external tissue surface of a patient, the external unit having at least one of (i) an optical source for wirelessly transmitting a light based signal transcutaneously and (ii) a receiver for receiving a light based signal transcutaneously; and,
an internal unit for positioning, in use, adjacent tissue in an internal body cavity of the patient, the internal unit having at least one of (i) said receiver for receiving light based signals from the external unit optical source, and (ii) said optical source for transmitting the light based signals to the external unit receiver.

2. The apparatus recited in claim 1 wherein the optical source comprises at least one light emitting diode.

3. The apparatus recited in claim 1 wherein the optical source emits light at a wavelength between 400 nm and 15,000 nm.

4. The apparatus recited in claim 1 wherein the optical source emits light at a wavelength between 700 to 1400 nm.

5. The apparatus recited in claim 1 wherein the receiver includes at least one filter for controlling the wavelength of the received light based signals.

6. The apparatus recited in claim 1 wherein the external unit has at least one said optical source; and,
the internal unit has at least one said receiver.

7. The apparatus recited in claim 1 wherein the internal unit has at least one said optical source; and,
the external unit has at least one said receiver.

8. The apparatus recited in claim 7 wherein the external unit further comprises at least one optical source for wirelessly transmitting a light based signal transcutaneously and the internal unit further comprises at least one receiver for receiving the light based signals from the external unit optical source.

9. The apparatus recited in claim 8 wherein the external unit has a plurality of receivers and further comprises a plurality of optical sources for wirelessly transmitting light based signals transcutaneously; and wherein the internal unit has a plurality of optical sources and further comprises a plurality of receivers;
wherein each of the plurality of receivers is optically configured for receiving the light based signals from a different one of the plurality of optical sources to define optically coupled pairs.

10. The apparatus recited in claim 9 wherein there are four optically coupled pairs.

11. The apparatus recited in claim 9 wherein the optical sources are light emitting diodes.

12. The apparatus recited in claim 11 wherein the plurality of optical sources emits light at wavelengths between 400 to 3000 nm.

13. The apparatus recited in claim 11 wherein the plurality of optical sources emits light at wavelengths between 750 to 1100 nm.

14. The apparatus recited in claim 9 further comprising an imaging device for generating video signals, wherein the light based signals emitted from the plurality of optical sources of the internal unit are video signals encoding a video image, and,
the plurality of receivers on the external unit is operatively connected to a video viewer for displaying the video image.

15. The apparatus recited in claim 14 further comprising a working instrument operatively connected to the internal unit, wherein the light based signals emitted from the external unit are control signals for controlling the working instrument.

16. The apparatus recited in claim 15 wherein the working instrument is a video camera.

17. An apparatus comprising:
an external unit for positioning, in use, on an external tissue surface of a patient, the external unit having (i) at least one optical source for wirelessly transmitting a light based signal transcutaneously and (ii) at least one receiver for receiving a light based signal transcutaneously; and,
an internal unit for positioning, in use, adjacent tissue in an internal body cavity of the patient, the internal unit having (i) at least one of said receiver for receiving light based signals from the external unit optical source and (ii) at least one of said optical source for transmitting the light based signals to the external unit receiver.

18. The apparatus recited in claim 17 wherein each receiver includes at least one filter for controlling the wavelength of the received light based signals.

19. The apparatus recited in claim 17 wherein each optical source emits light at wavelengths between 400 to 3000 nm.

20. A method for wirelessly transmitting a light based signal transcutaneously comprising:
inserting an internal unit into an internal body cavity of a patient undergoing a medical procedure;
positioning the internal unit adjacent tissue in an internal body cavity of the patient;
positioning an external unit on an external tissue surface of the patient opposite the position of the internal unit;
wherein the external unit has at least one of (i) an optical source for wirelessly transmitting a light based signal transcutaneously and (ii) a receiver for receiving a light based signal transcutaneously, and the internal unit has at least one of (i) said receiver for receiving light based signals from the external unit optical source, and (ii) said optical source for transmitting the light based signals to the external unit receiver;
transmitting light based signals from the external unit optical source to the internal unit receiver to effect operation of a working instrument operatively connected to the internal unit; and,
transmitting light based signals from the internal unit optical source to the external unit receiver to communicate information from the internal body cavity of the patient to a controller on the exterior of the patient.
